(19) United States
(12) Patent Application Publication
(10) Pub. No.: US 2010/0151395 A1
(43) Pub. Date: Jun. 17, 2010

(54) Protective film-removing solvent and method of photoresist patterning with it

(76) Inventors: Keita Ishiduka, Kawasaki-shi (JP); Masaaki Yoshida, Kawasaki-shi (JP); Satoshi Maemori, Kawasaki-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/656,768

(22) Filed: Feb. 16, 2010

Related U.S. Application Data
(63) Continuation of application No. 12/379,298, filed on Feb. 18, 2009.
Application No. 12/379,298 is a continuation of application No. 12/213,643, filed on Jun. 23, 2008, now abandoned.
Application No. 12/213,643 is a continuation of application No. 11/907,063, filed on Oct. 9, 2007, now abandoned.
Application No. 11/907,063 is a continuation of application No. 11/704,224, filed on Feb. 9, 2007, now abandoned.

(30) Foreign Application Priority Data
Feb. 10, 2006 (JP) 2006-034599

(51) Int. Cl. G03F 7/20; C07C 43/12

(52) U.S. Cl. 430/325; 568/683

(57) ABSTRACT

Disclosed are a protective film-removing solvent for re-moving a protective film laminated on a photoresist film, which contains at least a hydrofluoroether; and a method of photoresist patterning in liquid immersion lithography, using the protective film-removing solvent.

### BACKGROUND OF THE INVENTION

1. Field of the Invention

The present invention relates to a protective film-removing solvent for removing a protective film formed on a photoresist film, and to a method of photoresist patterning with it. In particular, the invention is favorably applied to a process of liquid immersion lithography.

2. Description of Related Art

Photolithography is much used in fabrication of micro-structures in various electronic devices, such as semiconductor devices, liquid-crystal devices. Recently, the increase in the technical level of large-scale integration and microfabrication is great, and it is desired to further improve the technique of photoresist micropatterning in photolithography.

At present, for example, photolithography in the forefront of the region of high-technology has made it possible to form a photoresist micropattern having a line width of 90 nm or so, and further studies and developments are being made for micropatterning to a higher level to a line width of 65 nm or so.

For attaining micropatterning to such a higher level, in general, some methods of improving exposure devices or photoresist materials may be taken into consideration. Regarding the method of improving exposure devices, there may be mentioned a method of employing short-wave light sources of F₂ excimer laser, EUV (extreme-ultraviolet ray), electron ray, X ray, soft-X ray, and a method of employing lenses having an increased numerical aperture (NA) . On the other hand, regarding the method of improving photoresist materials, there may be mentioned a method of developing a novel material exposable to short-wave light in processing it.

However, the method of employing such short-wave light sources requires an additional expensive exposure unit. On the other hand, the method of employing such increased-NA lenses is problematic in that, since the resolution and the focal depth range are in a trade-off relationship, the increase in the resolution may lower the focal depth range. Further, the development of a novel photoresist that may be exposed to short-wave light in processing it is expensive.

Recently, liquid immersion lithography has been reported as a technique of photolithography capable of solving these problems (for example, see the following References 1 to 3) . This method is for photoresist patterning by exposing to light a photoresist film formed on a substrate, in which, in the exposure light pathway space between the exposure device (lens) and the photoresist film, a liquid for liquid immersion lithography having a predetermined thickness is made to be onto at least the photoresist film, and the photoresist film is exposed to light in that condition to form a photoresist pattern. In the method of liquid immersion lithography, the exposure light pathway space, which is filled with an inert gas such as air or nitrogen in conventional methods, is substituted with a liquid for liquid immersion lithography, having a refractive index (n) larger than that of the space (vapor) and smaller than that of the photoresist film (for example, pure water and fluorine-containing inert liquid). The advantage of the method of liquid immersion lithography is that, even though an exposure light source having the same wavelength level as that in conventional methods is used therein, the method may attain a high-level resolution like the case that uses an exposure light having a shorter wavelength or Uses a high-NA lens and, in addition, the method does not result in the reduction in the focal depth range. In addition, in the method, photoresist materials generally used at present can be used.

To that effect, the process of liquid immersion lithography is much noticed in the art, since it realizes photoresist patterning of high resolution to a good focal depth at low costs, using any lens actually mounted on the existing exposure devices. therein.

However, in the process of liquid immersion lithography, exposure is performed while a liquid for liquid immersion lithography is put between an exposure lens and a photoresist film. Naturally, therefore, the method has some problems in that the photoresist film may be deteriorated by the liquid for liquid immersion lithography and the liquid for liquid immersion lithography itself may also be deteriorated by the component dissolved out of the photoresist film whereby the refractive index thereof may vary.

Given that situation; a technique has been proposed which comprises forming a protective film of a fluorine-containing resin (fluorine-substituted polymer) onto a photoresist film and disposing a liquid for liquid immersion lithography onto the protective film, and this is for the purpose of preventing the photoresist film from being deteriorated by the liquid for liquid immersion lithography, preventing the liquid for liquid immersion lithography from being deteriorated and thereby preventing the refractive index thereof from varying; and it has been confirmed that the protective film in the method gives a photoresist pattern having a good rectangular cross-sectional profile in liquid immersion lithography (for example, see the following. Reference 4). The protective film comprising such a fluorine-substituted polymer must be finally removed, but a special. removing solvent exclusive for the removal of the protective film comprising a fluorine-substituted polymer must be used, and a removing solvent that has better removing performance is desired. Furthermore, if the removing solvent is recyclable, then it is desirable from the viewpoint of global environment protection and of cost reduction.

Reference 1: Journal of Vacuum Science & Technology B, USA, 1999, Vol. 17, No. 6, pp. 3306-3309.

Reference 2: Journal of Vacuum Science & Technology B, USA, 2001, Vol. 19, No. 6, pp. 2353-2356,

Reference 3: Proceedings of SPIE, USA, 2002, Vol. 4691, pp. 459-465,

Reference 4: WO2004/074937.

### SUMMARY. OF THE INVENTION

The invention has been made in consideration of the above situation, and its object is to provide a protective film-removing solvent which may remove a protective film, especially a protective film comprising a fluorine-substituted polymer, in a simplified manner and efficiently and, in addition, which is recyclable; and to provide a method of photoresist patterning with the removing solvent.

In order to solve the above-mentioned problems, the invention provides a protective film-removing solvent which is for removing a protective film laminated onto a photoresist film and which contains at least a hydrofluoroether.

The invention also provides a method of photoresist patterning in liquid immersion lithography, which comprises providing a photoresist film on a substrate, forming a protective film onto the photoresist film, then disposing a liquid for liquid immersion lithography onto at least the protective film on the substrate, thereafter selectively exposing the photoresist film to light via the liquid for liquid immersion lithography and the protective film, then optionally heating it, and removing the protective film by the use of the above-mentioned protective film-removing solvent, and thereafter developing the photoresist film to thereby form a photoresist pattern.

### EFFECT OF THE INVENTION

The invention provides a protective film-removing solvent which may remove a protective film, especially a protective film comprising a fluorine-substituted polymer, in a simplified manner and efficiently and, in addition, which is recyclable. Applying the protective film-removing solvent of the invention to a process of liquid immersion lithography makes it possible to form an ultra-microfine photoresist pattern having a resolution much higher than that obtainable in conventional photolithography of a conventional photoresist material using a conventional exposure device.

### DETAILED DESCRIPTION OF THE INVENTION

The invention is described in detail hereinunder.

The protective film-removing solvent of the invention is characterized in that it contains at least a hydrofluoroether. The hydrofluoroether is a hydrofluorocarbon having an ether bond. the invention, in view of its washability and industrial producibility, the hydrofluoroether is preferably at least one selected from compounds with a hydrocarbon group having from 1 to 4 carbon atoms and a fluoroalkyl group having from 2 to 10 carbon atoms that bond to each other via an ether bond.

Concretely, examples of the hydrofluoroether of the type are C₂F₅OCH₃, C₂F₅OC₂H₅, C₂F₅OC₃H₇, C₂F₅OC₄H₉, C₃F₇OCH₃, C₃F₇OC₂H₅, C₃F₇OC₃H₇, C₃F₇OC₄H₉, C₄F₉OCH₃, C₄F₉OC₂H₅, C₄F₉OC₃H₇, C₄F₉OC₄H , C₅F₁₁OC₁₁OCH₃, C₅F₁₁OC₂H₅, C₅F₁₁OC₃H₇, C₅F₁₁OC₄H₉, C₆F₁₃OCH₃, C₆F₁₃OC₂H₅, C₆F₁₃OC₃H₇, C₆F₁₃OC₄H₉, C₇F₁₅OCH₃, C₇F₁₅OC₂H₅, C₇F₁₅OC₃H₇, C₇F₁₅OC₄H₉, C₈F₁₇OCH₃, C₈F₁₇OC₂H₅, C₈F₁₇OC₃H₇, C₈F₁₇OC₄H₉, C₉F₁₉OCH₃, C₉F₁₉OC₂H₅, C₉F₁₉OC₃H₇, C₉F₁₉OC₄H₉, C₁₀F₂₁OCH₃, C₁₀F₂₁OC₂H₅, C₁₀F₂₁OC₃H₇, C₁₀F₂₁OC₄H₉. These hydrocarbon groups and fluoroalkyl groups may have any of linear, branched or cyclic structures.

The hydrofluoroethers may be used singly or as a mixed solvent of two or more of them. The combination for the mixed solvent may be suitably determined depending on the physical properties of the constitutive hydrofluoroethers. For example, a high-boiling-point hydrofluoroether may be combined with a low-boiling-point hydrofluoroether, and the recovery of the resulting mixture in recycling the constitutive solvents may be increased while the mixture may keep its high film-stripping capability not diversifying the solvents.

Of the above-mentioned hydrofluoroethers, most preferred is at least one selected from C₄F₉OCH₃, C₄F₉OC₂H₅ and C₆F₁₃OCH₃.

The protective film-removing solvent of the invention is used in a process of photolithography, especially suitably for removing a protective film that comprises a fluorine-substituted polymer insoluble in water and alkali.

Concretely, the process of photolithography includes an ordinary photoresist patterning process and a process of photoresist patterning in liquid immersion lithography.

The protective film comprising a fluorine-substituted polymer includes those formed as a protective film for protecting an upper layer of photoresist from an external pollution factor, for example, in a photoresist patterning process including the above-mentioned cases in photolithography. The external pollution factor includes amine components in air that may cause deactivation of an acid to be generated by an acid-generating agent in a photoresist film in an ordinary photoresist patterning process; and in a photoresist patterning process in liquid immersion lithography, it includes the liquid for liquid immersion lithography to be disposed between a exposure lens and a photoresist film.

The fluorine-substituted polymer insoluble in water and alkali includes, for example, linear fluoroalkyl ether polymer, cyclic fluoroalkyl ether polymer, polychlorotrifluoroethylene, polytetrafluoroethylene, tetrafluoroethylene-perfluoroalkoxyethylene copolymer, tetrafluoroethylene-hexafluoropropylene copolymer, to which, however, the invention should not be limited.

Of the fluorine-substituted polymers mentioned above, preferred are linear fluoroalkyl ether polymer and cyclic fluoroalkyl ether polymer. Most preferred is a mixed polymer of cyclic fluoroalkyl ether polymer and linear fluoroalkyl ether polymer, or use of cyclic fluoroalkyl ether polymer alone. Linear fluoroalkyl ether polymer is commercially available as “Demnum S-20”, “Demnum S-65”, “Demnum S-100”, “Derfinum S-200” (all by Daikin); and cyclic perfluoroalkyl polyether is commercially available as “Cytop” series (by Asahi Glass), “Teflon-AF1600” and “Teflon-AF2400” (both by DuPont). These are favorably used in the invention.

The film that comprises the fluorine-substituted polymer is used preferably by dissolving the fluorine-substituted polymer in a fluorine-containing. organic solvent. Not specifically defined, the fluorine-containing organic solvent may be any one capable of dissolving a fluorine-substituted polymer. For example, its preferred examples are perfluorotributylamine, perfluorotetrapentylamine, perfluorotetrahexylamine.

Any other organic solvent and surfactant miscible with the fluorine-containing organic solvent may be suitably mixed with it.

In case where a fluorine-substituted polymer is dissolved in a fluorine-containing organic solvent to prepare a fluorine-substituted polymer-containing solution, the concentration of the solution may fall within a range capable of forming a film, not specifically defined. However, in consideration of the coatability with the solution, the concentration is preferably from 0.1 to 30% by mass or so.

The protective film-removing solvent of the invention is favorably used in liquid immersion lithography. The process of liquid immersion lithography applicable to the invention comprises forming a coating film (protective film) of a fluorine-substituted polymer insoluble in water and alkali, onto a photoresist film formed on a substrate, providing a liquid layer (for liquid immersion lithography) having a predetermined thickness between the protective film and an exposure device (lens), and exposing the photoresist film to light in that condition to thereby improve the resolution of the resulting photoresist pattern. After the exposure, the coating film (protective film) of a fluorine-substituted polymer is dissolved and removed away by the use of the removing solvent of the invention in a simplified manner and efficiently.

Concretely, the method of photoresist patterning in liquid immersion lithography using the protective film-removing solvent of the invention may be attained, for example, as follows:

First, an ordinary photoresist composition is applied on a substrate such as a silicon wafer, with a spinner, and then prebaked (for PAB treatment) to form a photoresist film thereon. An organic or inorganic antireflection film (undercoating antireflection film) may be previously formed on the substrate, and then a photoresist film may be formed thereon.

Not specifically defined, the photoresist composition may be any one developable with an aqueous alkali solution, including negative and positive photoresists.

Next, the above-mentioned, fluorine-substituted polymer-containing solution is uniformly applied onto the surface of the photoresist film, and then thermally cured to form a protective film (coating film) of the fluorine-substituted polymer.

Next, a liquid for liquid immersion lithography is disposed between the protective film and an exposure device (lens). In that condition, the photoresist film is selectively exposed to light through a mask pattern.

Accordingly, the exposure light shall reach the photoresist film, after passing through the liquid for liquid immersion lithography and the protective film.

In this process, the photoresist film is kept away from the liquid for liquid immersion lithography by the protective film formed thereon, and is therefore protected from the invasion by the liquid for liquid immersion lithography to be swollen or deteriorated, and on the contrary, the photoresist film is prevented from releasing its component into the liquid for liquid immersion lithography to change the optical properties such as the refractivity of the liquid itself for liquid immersion lithography.

The exposure light is not specifically defined, for which usable are any radiations such as ArF excimer laser, KrF excimer laser, F₂ excimer laser, EB, EUV, VUV (vacuum ultraviolet ray). The exposure light to be used may be selected and determined, essentially depending on the properties of the photoresist film.

Not specifically defined, the liquid for liquid immersion lithography may be any liquid having a refractive index larger than that of air and smaller than that of the photoresist film used. The liquid for liquid immersion lithography of the type includes, for example, water (pure water, deionized water), and fluorine-containing inert liquids. In addition, also usable herein is a liquid for liquid immersion lithography having high-refractivity characteristics, which may be developed in future. Specific examples of the fluorine-containing inert liquids are liquids comprising, as the principal ingredient thereof, a fluorine-containing compound such as C₃HCl₂F₅, C₄F₉OCH₃, C₄F₉OC₂H₅, C₅H₃F₇. Of those, preferred for the liquid for liquid immersion lithography is water (pure water, deionized water) from the viewpoint of the cost, the safety, the environmental problem and the wide-range applicability thereof. However, when an exposure light having a wavelength of 157 nm (e.g., F₂ excimer laser) is used, then preferred for it is a fluorine-containing solvent from the viewpoint that the absorption of the exposure light by the solvent is small.

After the step of exposure of the photoresist film kept immersed in the liquid as above, the liquid for liquid immersion lithography is removed away from the substrate.

Next, while the protective film is still onto the exposed photoresist film on the substrate, the substrate is subjected to PEB (post-exposure baking), and then the protective film-removing solvent of the invention is brought into contact with the substrate having the photoresist film and the protective film, thereby removing the protective film. For contacting it with the substrate, employable is any method of paddling, immersion or showering.

After the protective film is removed, the substrate having the photoresist film thereon is developed with an alkali developer comprising an aqueous alkali solution. The alkali developer may be any ordinary one. After the development, the substrate may be subjected to post-baking treatment. Next, the substrate is rinsed with pure water or the like. The rinsing with water may be effected, for example, as follows: While the substrate is rotated, water is dropped or sprayed on the surface of the substrate so that the developer and the photoresist composition dissolved by the developer are washed away. Then, this is dried, and a photoresist pattern reflecting the profile of the mask pattern used is thus formed on the substrate.

In that manner, a photoresist pattern is formed in the invention. Thus formed, the photoresist pattern has a micro-structure of good resolution, and in particular it may be a line-and-space pattern having a small pitch. The pitch of the line-and-space pattern as referred to herein means the total distance of the photoresist pattern width and the space width in the line width direction of the pattern.

The protective film-removing solvent of the invention is recyclable, and therefore has advantages of environmental protection and cost reduction.

Concretely, in the process of photolithography with the protective film-removing solvent of the invention, the used solvent is recovered after the washing treatment and then purified to obtain a recycled removing solvent; and the thus-recycled removing solvent may be again used for protective film removal in photolithography.

The recovered matter of the used solvent after the treatment of removing the fluorine-substituted polymer film (protective film) contains a fluorine-substituted polymer and a fluorine-containing organic solvent as impurities. For purifying the recovered matter, these impurities are removed through distillation, or that is, the impurities-containing fraction is removed, and the hydrofluoroether-containing fraction is collected, and then the thus-collected fraction is used as a recycled removing solvent.

### EXAMPLES

The invention is described in more detail with reference to the following Examples, to which, however, the invention should not be limited.

[Protective Film-Removing Solvent]

The following protective film-removing solvents were prepared.

Protective film-removing solvent 1: solvent comprising C₄F₉OCH₃.

Protective film-removing solvent 2: solvent comprising C₄F₉OC₂H₅.

Protective film-removing solvent 3: solvent comprising C₆F₁₃OCH₃. Protective film-removing solvent 4: mixed solvent of C₄F₉OC₂H₅ and C₆F₁₃OCH₃ (ratio by mass, 50/50).

### Example 1

In this Example, the ability of the protective film-removing solvent of the invention to dissolve and remove a protective film of a fluorine-substituted polymer is assessed as the basic property assessment of the protective film-removing solvent.

Concretely, a fluorine-substituted polymer-containing solution (protective film-forming material) was prepared by dissolving “Cytop CTX-809SP2” (by Asahi Glass) in perfluorotributylamine to have a concentration of 2.5% by mass, and this was applied onto a photoresist film formed on a substrate in a mode of spin coating therewith, and then heated at 90° C. for 60 seconds to form a protective film having a thickness of 28 nm.

The protective film was then kept in contact with any of the protective film-removing solvents 1, 2, 3 and 4, at 23° C. for 60 seconds, for removal of the protective film. In this treatment, the removing capability of the solvents is assessed by the time taken for the dissolution. As a result, all the solvents dissolved and removed the film within 40 seconds, and this means that the solvents all have good protective film-removing capability.

### Example 2

An organic antireflection film composition “ARC-29A” (by Brewer Science) was applied on a silicon wafer, using a spinner, and then baked and dried on a hot plate at 225° C. for 60 seconds to form an organic antireflection film having a thickness of 77 nm. On the antireflection film, applied was a positive photoresist “TArF-P6111ME” (by Tokyo Ohka Kogyo) using a spinner, and prebaked and dried on a hot plate at 130° C. for 90 seconds, thereby forming a photoresist film having a thickness of 225 nm on the antireflection film.

The above-mentioned, protective film-forming material was applied onto the photoresist film in a mode of spin coating with it, and then heated at 90° C. for 60 seconds to form a protective film having a thickness of 37 nm.

Next, through a mask pattern, this is exposed to patterning light of an ArF excimer laser (wavelength, 193 nm), using an exposure device “NSR-S302A” (by Nikon, NA (numerical aperture)=0.60, σ=2/3 ring) (exposure treatment) . After the exposure treatment, pure water was kept dropped onto the protective film at 23° C. for 2 minutes while the substrate was kept rotated, and this was thus kept in an environment of pseudo-liquid immersion lithography.

After the pure water-dropping step, this was subjected to PEB treatment at 130° C. for 90 seconds, and then the protective film was kept in contact with the removing solvent 1 at 23° C. for 60 seconds and removed. Next, this was developed with an aqueous 2.38 mas. % tetramethylammonium hydroxide (TMAH) solution at. 23° C. for 60 seconds to form a photoresist pattern.

The thus-formed, line-and-space (1/1) photoresist pattern having a pitch of 130 nm was observed with a scanning electronic microscope (SEM) The pattern had a good rectangular profile, and the removing treatment had no negative influence on the patterning.

### Example 3

In the same manner as in Example 2, an organic antireflection film composition “ARC-29A” (by Brewer Science) was applied on a silicon wafer, using a spinner, and baked and dried on a hot plate at 225° C. for 60 seconds to form an organic antireflection film having a thickness of 77 nm. Then, a positive photoresist “TArF-P6111ME” (by Tokyo Ohka Kogyo) was applied onto the antireflection film, using a spinner, and prebaked and dried on a hot plate at 130° C. for 90 seconds, thereby forming a photoresist film having a thickness of 150 nm on the antireflection film.

The above-mentioned, protective film-forming material was applied onto the photoresist film in a mode of spin coating with it, and then heated at 90° C. for 60 seconds to form a protective film having a thickness of 37 nm.

Next, using a laboratory kit for liquid immersion lithography (LEIES 193-1, by Nikon), the sample was tested for two-beam interference. Next, this was subjected to PEB treatment at 130° C. for 90 seconds, and then the protective film was kept in contact with the removing solvent 4 at 23° C. for 60 seconds and removed. This was subsequently developed with an aqueous 2.38 mas. % TMAH solution at 23° C. for 60 seconds, thereby forming a photoresist pattern.

Thus obtained, the line-and-space photoresist. pattern (1/1) having a pitch of 130 nm was observed with a scanning electronic microscope (SEM). The pattern had a good rectangular profile, and the removing treatment had no negative influence on the patterning.

### INDUSTRIAL APPLICABILITY

The protective film-removing solvent of the invention may efficiently dissolve and remove a protective film formed onto a photoresist film, especially a protective film comprising a fluorine-substituted polymer, and in addition, it is recyclable. The protective film-removing solvent of the invention may be applicable to a process of liquid immersion lithography, thereby forming an ultra-microfine photoresist pattern having a resolution much higher than that obtainable in conventional photolithography of a conventional photoresist material using a conventional exposure device.

What is claimed is:

1. A protective film-removing solvent for removing a protective film laminated on a photoresist film, which contains at least a hydrofluoroether.

2. The, protective film-removing solvent as claimed in claim 1, wherein the protective film is formed of a protective film-forming material to be used in a process of liquid immersion lithography.

3. The protective film-removing solvent as claimed in claim 1, wherein the protective film is a coating film of a fluorine-substituted polymer insoluble in water and alkali.

4. The protective film-removing solvent as claimed in claim 3, wherein the fluorine-substituted polymer contains at least a cyclic fluoroalkyl ether polymer.

5. The protective film-removing solvent as claimed in claim 1, wherein the hydrofluoroether is at least one selected from compounds with a hydrocarbon group having from 1 to 4 carbon atoms and a fluoroalkyl group having from 2 to 10 carbon atoms that bond to each other via an ether bond.

6. The protective film-removing solvent as claimed in claim 1, wherein the hydrofluoroether is at least one selected from C₄F₉OCH₃, C₄F₉OC₂H₅ and C₆F₁₃OCH₃.

7. A method of photoresist patterning in liquid immersion lithography, which comprises providing a photoresist film on a substrate, forming a protective film onto the photoresist film, then disposing a liquid for liquid immersion lithography onto at least the protective film of the substrate, thereafter selectively exposing the photoresist film to light via the liquid for liquid immersion lithography and the protective film, then optionally heating it, and removing the protective film by the use of the protective film-removing solvent of claim 1, and thereafter developing the photoresist film to thereby form a photoresist pattern.
